(19) United States
(12) Patent Application Publication
(10) Pub. No.: US 2017/0216571 A1
(43) Pub. Date: Aug. 3, 2017

(54) METHOD OF INCREASING THE LEAK TIGHTNESS OF A MECHANICAL CONNECTOR

(71) Applicant: FRESENIUS MEDICAL CARE DEUTSCHLAND GMBH, Bad Homburg (DE)

(72) Inventors: Ralf WOJKE, Bad Homburg (DE); Paul WIENEKE, Muenster (DE)

(73) Assignee: FRESENIUS MEDICAL CARE DEUTSCHLAND GMBH, Bad Homnurg (DE)

(21) Appl. No.: 15/501,554

(22) PCT Filed: Aug. 4, 2015
(86) PCT No.: PCT/EP2015/001606

§ 371 Date: Feb. 3, 2017

(30) Foreign Application Priority Data
Aug. 5, 2014 (DE) 10 2014 011 674.3

(51) Int. Cl. A61M 39/10; A61M 5/36; A61M 1/26

(52) U.S. Cl. CPC A61M 39/10; A61M 1/267; A61M 5/36; A61M 2039/1033; A61M 2039/1066; A61M 2205/0222

(57) ABSTRACT

The invention relates to a method of increasing the leak tightness of a mechanical connector of an extracorporeal blood treatment machine, wherein the mechanical connector has a pair of connection parts which have corresponding sealing surfaces, with at least one of the two sealing surfaces being wetted at least sectionally by a viscous fluid before the joining together of the connection parts and/or with the connection gap between the connection parts being covered by a sheath after their joining together. The invention further relates to an extracorporeal blood treatment machine comprising a mechanical connector which has a pair of connection parts which have corresponding sealing surfaces, with the sealing surfaces being at least sectionally wetted with a viscous liquid with a closed connector and/or with the connection gap between the connection parts of the closed connector being covered by a sheath.

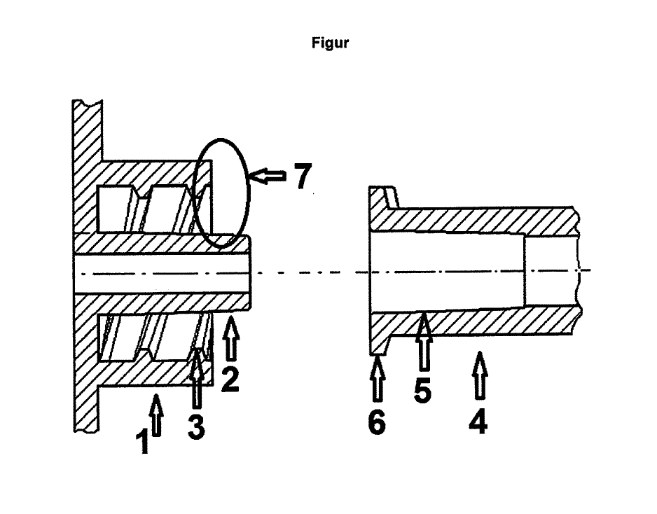

The invention relates to a method of increasing the leak tightness of a mechanical connector of an extracorporeal blood treatment machine as well as to an extracorporeal blood treatment unit.

Microbubbles are gas bubbles having a diameter of a few μm which are as a rule no longer visible due to their small size. They arise at different points and under different conditions in extracorporeal blood circuits, inter alia by the discharge of blood-soluble gases or by air entry at very small leaks in the vacuum region of the extracorporeal circuit, and adhere to the inner surface of the extracorporeal hose system or of the blood treatment unit. Their effects when they are detached and infuse into the human body are greater than previously presumed. For example, microbubbles were found in vital organs such as the lung, heart and brain of dialysis patients.

There are indications that, for example, the Luer-Lock connection between an arterial cannula and the arterial hose, a connection of two conical hard plastic parts which is located in the vacuum region of the blood pump, is not always airtight and small amounts of air are sucked into the blood hose. In a Luer-Lock connector, a hard plastic is pressed against a hard plastic, with the sealed region of such an areal pressing being limited to a cone. Investigations have confirmed that microbubble numbers and volumes are much larger downstream of a Luer-Lock connector than upstream of a Luer-Lock connector in the vacuum region.

Microbubbles can only be separated with limitations in the venous chamber due to their small size and their small buoyancy and can only be conditionally recognized by the prescribed protection system for avoiding air infusion.

It is the underlying object of the invention to reduce the number of microbubbles in extracorporeal blood circuits or to reduce the infusion of microbubbles into the patient's body.

Against this background, the invention relates to a method of increasing the leak tightness of a mechanical connector of a medical device, wherein the mechanical connector has a pair of connection parts which have corresponding sealing surfaces, with at least one of the two sealing surfaces being wetted at least sectionally by a viscous liquid before the joining together of the connection parts and/or with the connection gap between the connection parts being covered by a sheath after their joining together. In accordance with the invention, the above-named object is achieved by an increase in the leak tightness of existing connectors.

The viscous liquid can, for example, have a dynamic viscosity of between 10² and 10⁵, preferably of between 10³ and 10⁴ mPa·s at 25° C. A liquid having such viscosity values is particularly suitable for a simple handling in the application and for an effective seal. The liquid is preferably sterile and has a high biocompatibility.

The viscous liquid should in particular be blood-compatible, i.e. an entry of a small quantity of the liquid into the blood of a patient should not be harmful.

The connection gap extends along the outer periphery of the contact surface between the two connection parts. The joining together comprises a contacting of the two sealing surfaces as well as a locking of the two connection parts in a manner such that the contact of the two sealing surfaces is maintained and the sealing surfaces preferably lie on one another with a certain contact pressure. This can be achieved, for example, by a screwing or latching of the two connection parts. The connection parts and in particular the contact surfaces of the connection parts can be produced from hard plastic in an embodiment.

In an embodiment, one connection part has a male cone and one connection part has a female cone, with at least one section of the outer jacket surface of the male cone and at least one section of the inner jacket surface of the female cone forming the sealing surfaces. The two respective sections contact one another after a joining together of the parts and seal the plug-in connection.

In an embodiment, the mechanical connector is a Luer-Lock connection. In this respect, it is a standardized connector for medical engineering, with one connection part of the connector having a male cone and the other connection pair having a female cone. To secure the connections against a release in an optimum manner, the Luer-Lock connector furthermore has a system for screwing the connection parts in addition to the cones, with an external thread at the connection part in which the female cone is located engaging into an internal thread which is seated at the connection part having the male cone.

In an embodiment, only the sealing surface of a connection part is wetted with the viscous liquid before the joining together of the connection parts. Provision can, for example, be made that only the jacket surface of the male cone is wetted with the viscous liquid. This is sufficient for establishing an air-impermeable connection and the outer jacket surface of the male cone is more easily accessible than the inner jacket surface of the female cone.

In an embodiment, the medical device is an extracorporeal blood treatment machine. The extracorporeal blood treatment machine can be a dialyzer. Furthermore, the extracorporeal blood treatment machine can, for example, be an ultrafiltration device or a heart-lung machine. The use of the method in accordance with the invention is also conceivable in other medical devices. For example, the use is also conceivable in endoscopic abdominal surgery or in gynecology, with an artificial surgical cavity being able to be created with the aid of gases. A gas-impermeable connection would be very meaningful here to limit the introduced quantity of gas.

In an embodiment, the connector at which the method is implemented connects a liquid-conducting circuit to an inflow line or outflow line. For example, the liquid-conducting circuit can be an extracorporeal blood circuit. This circuit is typically configured as a disposable, with the disposable having different interfaces which are connected to lines, cannula, etc. at the machine side with the aid of a connector. Examples for inflow lines and outflow lines in the sense of this embodiment comprise the arterial port, a predilution line, a postdilution line, a heparin line, a port for blood removal, a port for medication dispensing, a drain or the venous port. Provision can in particular be made that the connector at which the method is implemented connects an arterial cannula to the arterial line of an extracorporeal blood circuit (arterial port).

In an embodiment, the liquid-conducting circuit comprises at least one pump and the method is implemented at a connector which is arranged on the intake side of this at least one pump. Air can in particular enter into the circuit at the intake side of the pump since there is a vacuum with respect to the environment. For example, the liquid-conducting circuit is an extracorporeal blood circuit and the method is implemented at a connector which is arranged on the intake side of the blood pump.

In an embodiment, the sheath is a flexible sheath. A tighter-sealing connection can be achieved with the aid of a flexible sheath than with a rigid protector since the sheath can nestle against the outer surface of the connector. Furthermore, a flexible sheath can be manufactured less expensively and can be simply slipped over the connector after the joining together of the connection parts. Provision can be made that the sheath is slipped over the connector such that it completely covers the region of the connection gap.

In an embodiment, the sheath is a flexible hose piece or a ring. These shapes make it possible simply to push the sheath over the connector after the joining together of the connection parts.

In an embodiment, the sheath is produced from a silicone elastomer. This material has proved particularly suitable due to the good properties with respect to elasticity, biocompatibility and leak tightness.

The invention further relates to a medical device comprising a mechanical connector which has a pair of connection parts which have corresponding sealing surfaces, with the sealing surfaces being at least sectionally wetted with a viscous liquid with a closed connector and/or with the connection gap between the connection parts of the closed connector being covered by a sheath.

The closed connector designates the state of the connector after the joining together of the connection parts. The medical device can be an extracorporeal blood treatment machine. The extracorporeal blood treatment machine can be a dialyzer. Furthermore, the extracorporeal blood treatment machine can, for example, be an ultrafiltration device or a heart-lung machine. It is furthermore conceivable that the medical device is, for example, an instrument of endoscopic abdominal surgery or of gynecology. Further medical devices at which a gas-impermeable connection of two connection parts is meaningful are also covered by the present invention.

In an embodiment, one connection part has a male cone and one connection part has a female cone, with at least one section of the outer jacket surface of the male cone and at least one section of the inner jacket surface of the female cone forming the sealing surfaces. In an embodiment, the mechanical connector is a Luer-Lock connection.

In an embodiment, the connector connects a liquid-conducting circuit, in particular an extracorporeal blood circuit, to an inflow line and an outflow line. For example, the liquid-conducting circuit can be an extracorporeal blood circuit. This circuit is typically configured as a disposable, with the disposable having different interfaces which are connected to lines, cannula, etc. at the machine side with the aid of a connector. Examples for inflow lines and outflow lines in the sense of this embodiment comprise the arterial port, a predilution line, a postdilution line, a heparin line, a port for blood removal, a port for medication dispensing, a drain or the venous port. In an embodiment, the liquid-conducting circuit comprises at least one pump and the connector is arranged on the intake side of this at least one pump. The liquid-conducting circuit is, for example, an extracorporeal blood circuit and the connector is arranged on the intake side of the blood pump. Provision can in particular be made that the connector connects an arterial cannula to the arterial line of an extracorporeal blood circuit (arterial port).

In an embodiment, the sheath is a flexible sheath, with provision preferably being made that this sheath completely covers the region of the connection gap. The sheath can be a flexible hose piece or a ring. The sheath can be produced from a silicone elastomer.

Further details and advantages result from the FIGURES and embodiments described in the following. The only FIGURE shows an illustration of a Luer-Lock connector in cross-section.

The connector has a first connection part 1 having a male cone 2 and an internal thread 3. The second connection part 4 has a female cone 5 and a projection 6 which engages into the internal thread. On the joining together of the connection parts 1 and 4, they are screwed and the outer jacket surface of the male cone 2 and the inner jacket surface of the female cone 5 are pressed against one another.

Provision is made in accordance with the invention that the outer jacket surface of the male cone 2 is wetted with a viscous, sterile and biocompatible liquid before the joining together of the two connection parts 1 and 4.

Provision is alternatively or additionally made in accordance with the invention that a hose piece of a flexible silicone elastomer is slipped over the connector after the joining together of the connection parts 1 and 4. The inner diameter of the flexible hose piece or of the ring is selected somewhat smaller than the outer diameter of the connector to achieve an airtight termination. The hose piece should in particular completely cover the connection gap which arises at the point marked by reference numeral 7 between the two connection parts after their joining together.

1. A method of increasing the leak tightness of a mechanical connector of a medical device, in particular of a dialysis machine, wherein the mechanical connector has a pair of connection parts which have corresponding sealing surfaces,
characterized in that at least one of the two sealing surfaces is at least sectionally wetted with a viscous liquid before the joining together of the connection parts; and/or in that the connection gap between the connection parts is covered by a sheath after their joining together.

2. A method in accordance with claim 1, characterized in that one connection part has a male cone and one connection part has a female cone, with at least one section of the outer jacket surface of the male cone and at least one section of the inner jacket surface of the female cone forming the sealing surfaces.

3. A method in accordance with claim 1, characterized in that the mechanical connector is a Luer-Lock connector.

4. A method in accordance with claim 1, characterized in that only the sealing surface of a connection part, preferably only the jacket surface of the male cone, is wetted with the viscous liquid before the joining together of the connection parts.

5. A method in accordance with claim 1, characterized in that the connector at which the method is implemented connects a liquid-conducting circuit, in particular an extracorporeal blood circuit, to an inflow or to an outflow.

6. A method in accordance with claim 5, characterized in that the liquid-conducting circuit comprises at least one pump and the method is implemented at a connector which is arranged on the intake side of this at least one pump.

7. A method in accordance with claim 1, characterized in that the sheath is a flexible sheath, with provision preferably being made that this sleeve is slipped over the connector such that it completely covers the region of the connection gap.

8. A method in accordance with claim 1, characterized in that the sheath is a flexible hose piece or a ring.

9. A method in accordance with claim 1, characterized in that the sheath is produced from a silicone elastomer.

10. A medical device, in particular a dialysis machine, comprising a mechanical connector which has a pair of connection parts which have corresponding sealing surfaces,
characterized in that the sealing surfaces are wetted with a viscous liquid at least sectionally with a closed connector; and/or in that the connection gap between the connection parts of the closed connector is covered by a sheath.
